(19) United States
(12) Patent Application Publication
(10) Pub. No.: US 2010/0198025 A1
(43) Pub. Date: Aug. 5, 2010

(54) METHOD AND ARRANGEMENT FOR UNAFFECTED MATERIAL ANALYSE

(76) Inventors: Olof Lindahl, Umea (SE); Kerstin Ramser, Lulea (SE)

Correspondence Address: NIXON & VANDERHYE, PC, 901 NORTH GLEBE ROAD, 11TH FLOOR, ARLINGTON, VA, 22203, US

(21) Appl. No.: 12/733,810

(22) PCT Filed: Sep. 11, 2008
(86) PCT No.: PCT/SE2008/051018

§ 371 Date: Mar. 22, 2010

(30) Foreign Application Priority Data
Oct. 1, 2007 (SE) 0702207-2

(51) Int. Cl. A61B 5/00

(52) U.S. Cl. 600/301

(57) ABSTRACT

This invention concerns a method for the unaffected material analyse for detection and analysis of at least one material deviation in a material 2. The method is characterised by the detection of a region 1 with a difference in hardness in the material 2 through the use of a tactile sensor 4 that is placed in contact with the material, illumination of the detected area with chromatographic light A in order to obtain reflected light B in the form of a light spectrum, and analysis of the obtained light spectrum in order to obtain information concerning the physical and chemical molecular structure of the material. The invention concerns also an arrangement.

This invention concerns a method and an arrangement for unaffected material analyse.

It is desirable and necessary within many technical areas to investigate material and its properties, in order, for example, to detect, localise and determine at least one of changes, deviations, variations, differences, defects and similar in the material and to check the distributions of material, both desired and undesired.

Such material analysis has previously occurred through the material being cut and visually investigated, or through parts of the material being removed, samples of the material having been taken, and investigated in, for example, a microscope. This mechanical processing that occurs during such a material sampling normally destroys the material in question, at least in that part of it from which the sample is taken, or it causes or initiates other types of material change, or risks that undesired material changes take place.

The material may be a manufactured material or a natural material. The material may be an inorganic material or an organic material, such as living tissue.

One purpose of this invention is to offer a method and an arrangement that make it possible to carry out a unaffected material analyse for the detection and analysis of at least one material deviation, variation, difference or similar, in a material.

This purpose is achieved with a method having the technical characteristics that are made clear by claim 1 and an arrangement having the technical characteristics that are made clear by claim 9.

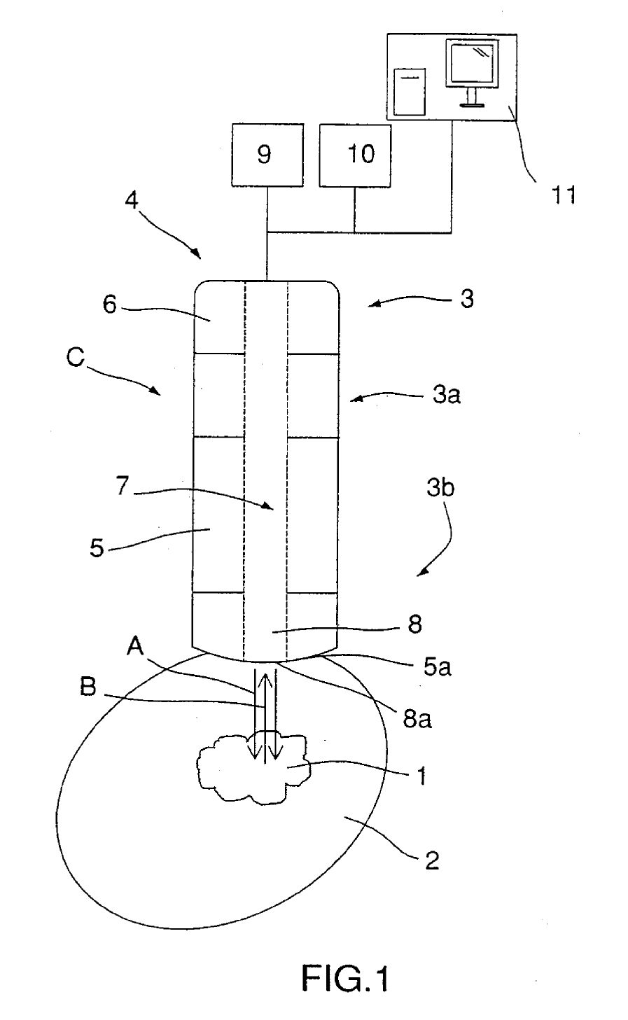

The invention will be described here with reference to the attached drawing. FIG. 1 shows a schematic FIGURE of an arrangement according to the invention.

The basic idea of the invention is to make it possible to combine the detection of a harder or softer region 1 in a material 2 with a material analysis of the detected region that takes place directly. This is to be carried out directly in the material without the need for any form of sampling, without the need that any material be removed. This leads to the influence on the material being kept to a minimum and it makes it possible to avoid future changes in the material as a result of the removal of material. This will be referred to below as “unaffected material analyse”. It is also a basic idea that it should be possible to carry out this method with the aid of one and the same arrangement 3.

A method according to the invention is intended to be used during unaffected material analyse for the detection and analysis of at least one region 1 that has a different hardness than that of the material otherwise, a material deviation, in a material 2 for which the basic properties, the desired properties, are known.

The method comprises the detection of a region 1 with a different hardness, stiffness, than the known material, a region 1 with a difference in hardness from that of the material otherwise. The detection of the region 1 takes place through the use of a tactile sensor 4, which is placed in contact with the material 2. The tactile sensor 4 reads differences in hardness, and these differences are recorded and analysed. This may take place either manually or by machine; it may be activated or automatic; there may be different recording systems, processing systems, feedback systems, reporting systems and similar used; and the arrangements associated with these may be different.

When a region 1 with a deviating hardness, stiffness, is detected, the detected region 1 is illuminated with chromatographic light A. The energy of the chromatographic light is partially transferred to the material in the detected region 1. The chromatographic light that is reflected from the region 1 has transferred energy to the material in the region 1. A light spectrum is obtained by recording the change in energy.

The method comprises also the analysis of the light spectrum that has been obtained in order to obtain information concerning the physical and chemical molecular structure of the material that is present in the detected region. The reflection will demonstrate a specific appearance, a specific light spectrum, that depends on the identity of the material.

The analysis may take place either manually, by man, or by machine; it may be either activated or automatic. Depending on the technical area in which the method is being used, it is appropriate that the light spectrum that has been obtained be compared with light spectra from common substances and from substances within the technical area.

The detection of the region 1 with a different hardness takes place by causing contact to be made with the tactile sensor 4, which is appropriately a resonance sensor. The main component of a resonance sensor is an element 5, a piezoelectric element, a ceramic, which is caused to vibrate, to oscillate, approximately in the same way as a vibrating guitar string, with the aid of an electrical circuit. The element 5 vibrates with a particular frequency, its resonance frequency. When a load, for example a material 2, is subsequently placed in contact with one end of the vibrating element 5, the acoustic impedance of the material will influence the vibrating element, the oscillating system, such that it vibrates at a new resonance frequency. The frequency at which the element 5 vibrates is determined by the hardness, the stiffness, of the material with which the sensor is in contact. If the sensor 4 is moved across a material 2, continuous changes in frequency can be recorded, and one or several regions 1 with different hardnesses can be detected and localised. The frequency changes, the differences in frequency, that are recorded, registered, depend on the hardness, the stiffness, of the material with which the sensor is in contact.

Thus, the method according to the invention comprises the activation of an electrical circuit 6 in order to cause the element 5, comprised within the resonance sensor 4, to vibrate in order to obtain a resonance frequency, and the placing of the element 4 in contact with the material 2, after which a difference in resonance frequency of the element can be measured and correlated with the hardness of the material with which the sensor is in contact, whereby a region 1 with a different hardness can be detected.

The method comprises the use of laser light in order to obtain the chromatographic light. It is appropriate and advantageous to use a Raman spectrometer 7, a Raman sensor, with a probe 8 that is placed in contact with the material. The Raman spectrometer 7 comprises a source 9 of laser light for illumination, and arrangements and units for the analysis. The use of such a sensor has proved to be valuable in the detection of cancerous changes in tissues. Tumours can be revealed since these usually demonstrate another chemical composition than that of healthy tissue, such as prostate tissue. Tumours give rise to a changed molecular composition of the tissue, and this is reflected in the spectrum that is recorded by the Raman spectrometer 7.

An arrangement 3 according to the invention is an arrangement 3 that makes unaffected material analyse possible, and that it is possible to use for the method. The arrangement comprises a tactile sensor 4 that is placed in contact with the material 2 for the detection of at least one region 1 with a difference in hardness compared with the known material 2. The tactile sensor 4 is a resonance sensor comprising an element 5 and an electrical circuit that is activated and that causes the element to vibrate in order to obtain a resonance frequency. The element 5 is a piezoelectric element.

The arrangement 3 comprises further an arrangement 9, a source of laser light, that emits chromatographic light and that illuminates the detected region 1 with the light A in order to receive reflected light B that can be reproduced as a light spectrum, and finally an arrangement 10 that comprises a detector (not shown in the drawing) and that analyses the light spectrum that has been received and that gives information about the material in the region, its physical and chemical molecular structure.

It is appropriate that the arrangement 3 comprise a Raman spectrometer 7, which in turn comprises a probe 8 that constitutes the end of an optical fibre through which the laser light propagates, the source 9 of laser light and the arrangement 10 for analysis and information processing. The arrangement 10 for analysis and information processing may be more or less manual or machine-based; it may be activated or automatic.

This invention, both the method and the arrangement, have principally been developed and tested within the technical area of medicine, where the unaffected material analyse has been carried out on living tissue, for the diagnosis of tumours, cancer tumours, in tissue, in prostate glands in vivo.

Histopathology is a common method for demonstrating the presence of cancer in tissue. It involves the detection and confirmation of the presence of morbid tissue changes in vitro, outside of the living organism, normally with a microscope. Histopathology is a time-consuming method that requires skilled personnel who are able to carry out correct sampling, normally in the form of a biopsy in which a tissue sample is taken with the aid of an instrument that is introduced into the tissue, and who are able to investigate, analyse and evaluate the sample, and interpret the result correctly.

It may be problematical to use this known method in certain situations, for example in cases of prostate cancer, which is the most common form of cancer affecting men in the USA and Europe. It is often not possible to locate tumours in the prostate with palpitation, analyse using the hands, nor with any commercially available imaging method, such as ultrasound, and this makes further sampling and analysis both uncertain and problematical.

Palpitation of the prostate takes place through a physician investigating the hardness of the tissue of the prostate using the fingers, via the rectum of the patient. The physician is seeking harder areas, since it is normally the case that tumours are harder than the surrounding healthy tissue. Even if the physician can feel harder areas and suspects the presence of cancer, it is difficult to determine the exact location of the tumour in the prostate.

In order to determine the location of any possible cancer tumour, it is necessary to take biopsies from several random locations in the prostate in order to obtain a clearer image of the location and extent of the cancer. The taking of biopsies entails creating wounds in skin and tissue and this increases the risk of complications arising, for example in the form of infections, since this type of sampling takes place in an area rich in bacteria close to the anus. It is also difficult to carry out the final analysis of the sample that has been taken. It has proved to be the case that cancer that is present is relatively often not detected. It has been estimated that this occurs as often as for 3 out of every 10 biopsies carried out.

The fact that the histopathologic analyses are carried out in vitro and cannot be carried out in vivo, in the living organism, since the sample that is to be analysed must be removed from the body, entails further risks for erroneous assessment of the sample of material, and of the disease condition related to it, since there is a risk that the sample is subjected to both mechanical and chemical changes during its removal from the body.

The main component of a resonance sensor is an element, a piezoelectric element, a ceramic, which can be caused to vibrate, approximately in the same way as a vibrating guitar string, with the aid of an electric circuit. The element vibrates with a particular frequency, its resonance frequency. When a load, for example a material, is subsequently placed in contact with one end of the vibrating element, the acoustic impedance of the material will influence the oscillating system such that it vibrates at a new resonance frequency. The frequency at which the element vibrates is determined by the hardness, the stiffness, of the material with which the sensor is in contact. If the sensor is moved across a material, continuous changes in frequency can be recorded, and one or several regions with different hardnesses can be detected and localised. The frequency changes, the differences in frequency, that are recorded, depend on the hardness, the stiffness, of the material with which the sensor is in contact.

The use of such a sensor has proved to be valuable in the detection of cancerous changes in tissues. Tumours can be revealed since these are usually harder than healthy tissue, such as prostate tissue.

Raman spectroscopy is a light-based method in which the material is illuminated with monochromatic light, normally laser light. The monochromatic light that impinges upon a material, a sample, causes motion in the illuminated molecules in the material and gives rise to changes in wavelength of the light, across the range of wavelengths that can be detected, and portions of the light are reflected back in the form of a spectrum, a spectrum of colours.

This spectrum is interpreted and provides detailed information about the molecular composition of the material and on the properties of the molecules.

An arrangement 3, an instrument, according to the invention combines two detection technologies in order not only to increase the diagnostic reliability but also to be able to provide supplementary information about which type of cell change is involved. The diagnosis will be better and more reliable since it is possible with one and the same arrangement to detect the presence of a material deviation, a tumour, and to analyse, investigate, it at the same time. Furthermore, the risk of infection, which is relatively common for sampling in which parts of the material, the tissue, are removed, is reduced.

The method and the arrangement 3 can be applied in other technical areas in which the components of a material have been accumulated to structures with a different hardness than the desired material, or where the material has acquired for one of various reasons accumulations of another material or several other materials of different hardness than the desired material. It may also be the case that the accumulated material is softer than the known surrounding material. The description that is presented here can be easily adjusted such that it is valid also for unaffected material analyse, inspection, where the material is not living tissue.

An arrangement 3 according to the invention can be made to be relatively small. It is currently possible to place a tactile sensor 5 and a Raman spectrometer probe 8 into one and the same arrangement body C. The arrangement body C can be held in one hand. The arrangement of the Raman spectrometer 9 that emits chromatographic light and an arrangement 10 that carries out analysis can also be arranged in the body, or these may be located fully or partially outside of the body itself, being placed in connection with other functions.

It is appropriate that the construction is such that the tactile sensor 5, in the form of a resonance sensor, is arranged around a Raman probe 8, which is then located in the centre. The resonance sensor 5 and the Raman probe 8 should have surfaces 5a and 8a of contact in the same plane such that it will be possible to place the arrangement against the material, and such that it is possible to use both the resonance sensor 5 and the Raman spectrometer 7 during the same occasion of contact.

The body C of the arrangement 3 can have the form of a pen with an extended body 3a that offers a region that can be gripped and held by one hand of the person who is carrying out the analyse. The arrangement should have a part 3b, a point, that can be placed against the material 2 in a firm and clear manner.

It is appropriate that electrical connections, connection arrangements and such, take place in the normal manner using cables or other feed arrangements, or both. The arrangement 3 can be connected in various ways to a computer 11 in which the measured values obtained can be stored, analysed and processed, and compared with other related data.

The arrangement 3, the instrument, is of major benefit during, for example, cancer surgery since a tumour will be well-defined both in terms of its extent and type, and this makes it possible for the surgeons to remove the cancer or tumour completely, without the need to remove quantities of healthy tissue in order to be on the safe side.

There are many applications for an arrangement, an instrument, according to the invention. There is a potential from a medical point of view that large parts of the human body can be analysed, investigated. A gastroscope, which is a long and flexible instrument used to view inside the stomach and gastrointestinal tract, being equipped with this technology would be very powerful and it would be possible to use this gastroscope for many different diagnosis processes and analyses. The method and the arrangement can also be used for the diagnosis of other forms of cancer, for example skin cancer and breast cancer.

1. A method for unaffected material analyse for detection and analysis of at least one material deviation in a material (2) characterised by detection of a region (1) with a difference in hardness in the material (2) through the use of a tactile sensor (4) that is placed in contact with the material, illumination of the detected region with chromatographic light (A) in order to obtain reflected light (B) in the form of a light spectrum, and analysis of the obtained light spectrum in order to obtain information concerning the physical and chemical molecular structure of the material.

2. A method according to claim 1, in which the detection of the region (1) with differences in hardness takes place by placing the tactile sensor (4), which is a resonance sensor, in contact with it.

3. A method according to claim 2, in which an electrical circuit (6) is activated in order to cause an element (5), comprised within the resonance sensor (4), to vibrate, oscillate, in order to obtain a resonance frequency, and in which the element (5) is placed in contact with the material (2), after which a difference in resonance frequency of the element (5) can be measured and correlated to the hardness of the material (2) with which the sensor is in contact.

4. A method according to claim 3, in which the element (5) is a piezoelectric element.

5. A method according to claim 1, in which laser light is used in order to obtain the chromatographic light.

6. A method according to claim 1, in which a Raman Spectrometer (7) is used for the illumination and analysis.

7. A method according to claim 1, in which the unaffected material analyse is carried out on living tissue.

8. A method according to claim 7, in which the unaffected material analyse is carried out on living tissue in order to detect and analyse tumours.

9. An arrangement (3) that makes possible the unaffected material analyse for the detection and analysis of at least one material deviation in a material (2),
characterised by:
a tactile sensor (5) that is placed in contact with the material (2) for detection of at least one region (1) with a difference in hardness in the material (2), an arrangement (7, 8, 9) that emits chromatographic light (A) and that illuminates the detected region (1) with this light in order to obtain reflected light (B) in the form of a light spectrum, and
an arrangement (10) that analyses the obtained light spectrum and that gives information about the material in the region, its physical and chemical molecular structure.

10. An arrangement according to claim 9, in which the tactile sensor (4) is a resonance sensor.

11. An arrangement according to claim 9, in which the resonance sensor (4) comprises an element (5) and an electrical circuit (6) that is activated and that causes the element (5) to vibrate, oscillate, in order to obtain a resonance frequency.

12. An arrangement according to claim 11, in which the element (5) is a piezoelectric element.

13. An arrangement according to claim 9, comprising a source (9) of laser light in order to obtain the chromatographic light.

14. An arrangement according to claim 13, comprising a Raman spectrometer (7), which in turn comprises the source (9) of laser light.,

15. An arrangement according to claim 9, for use in unaffected material analyse of living tissue.

16. An arrangement according to claim 15, for detection and analysis of tumours.

17. An arrangement according to claim 9, in which the tactile sensor (4) and at least a part of the arrangement (9) that emits chromatographic light are arranged in a body that can be held by one hand.

18. An arrangement according to claim 17, in which the tactile sensor (4) is arranged around the arrangement (9) that emits chromatographic light, which is then located in the centre of the body.
